(19) United States
(12) Patent Application Publication
(10) Pub. No.: US 2007/0225778 A1
(43) Pub. Date: Sep. 27, 2007

(54) PDT apparatus with an addressable LED array for therapy and aiming

(76) Inventors: Gregory Lee Heacock, Auburn, WA (US); William Louis Barnard, Maple Valley, WA (US); Wes Alan Williams, Silverdale, WA (US); Wayde Hampton Watters, Kent, WA (US)

Correspondence Address: MCANDREWS HELD & MALLOY, LTD, 500 WEST MADISON STREET, SUITE 3400, CHICAGO, IL, 60661, US

(21) Appl. No.: 11/387,995

(22) Filed: Mar. 23, 2006

(51) Int. Cl. A61N 5/06

(52) U.S. Cl. 607/88; 606/4; 607/90

(57) ABSTRACT

An apparatus for directing light to an eye for exciting a photosensitizer includes an addressable LED array so that the size and shape of the LED light used for aiming or therapy is selectable so as to match the size and shape of targeted tissue. The LED array has a configuration that provides increased LED density, increased current spreading with minimal light blocking and a simplified addressing connection sheme.

### CROSS-REFERENCE TO RELATED APPLICATIONS

N/A

### STATEMENT REGARDING FEDERALLY SPONSORED RESEARCH OR DEVELOPMENT

N/A

### TECHNICAL FIELD

The present invention relates to an apparatus for directing light to an eye for exciting a photosensitizing agent or photosensitizer to provide therapy for an ocular disease and more particularly to such an apparatus that includes an addressable LED array so that the size and shape of the LED light used for aiming or therapy is selectable so as to match the size and shape of targeted tissue.

### BACKGROUND OF THE INVENTION

Photodynamic Therapy (PDT) is a known process in which light of a specific wavelength or waveband is directed to tissues undergoing treatment or investigation that have been rendered photosensitive through the administration of a photo-reactive or photosensitizing agent called a photosensitizer. In this therapy, a photosensitizer having a characteristic light absorption waveband is first administered to the patient, typically either orally or by injection or even by local delivery to the treatment site. Proliferating cells, such as those involved in many eye diseases, may preferentially take up or absorb a number of photosensitizers. Once the drug or photosensitizer has been administered and reaches the target tissue, the tissue is illuminated with light of an appropriate wavelength or waveband corresponding to the absorption wavelength or waveband of the photosensitizer.

The object of the PDT may be diagnostic, where the energy level and wavelengths of light are selected to cause the photosensitizer to fluoresce, thus yielding information about the tissue without damaging the tissue. The object of the PDT may also be therapeutic, where the wavelength of light delivered to the photosensitive tissue under treatment causes the photosensitizer to undergo a photochemical interaction with oxygen in the tissue under treatment yielding free radical species such as a singlet oxygen, causing local tissue affect.

Typically, the light source used to excite the photosensitizer in PDT is a laser. However, the laser equipment used for PDT is relatively expensive. As an alternative, a non-coherent light source, such as an LED, has been used in PDT as described in U.S. Pat. No. 6,319,273. In order to select the size and shape of the LED light used for therapy in that system, a user selects a filter having an opaque region and a transparent region that determines the shape of the light used for therapy. In order to change the shape of the light the physician has to manually remove one filter and replace it with another filter. This is a time consuming process for the physician. Moreover, because the shape of the transparent region is fixed, the light cannot be tailored to match the shape of the diseased tissue. As such, these filters do not prevent healthy tissue from being irradiated with the therapy light.

### BRIEF SUMMARY OF THE INVENTION

In accordance with the present invention, the disadvantages of prior PDT systems and apparatus for treating ocular diseases have been overcome. In accordance with the present invention, the PDT apparatus includes an addressable LED array so that the size and shape of the LED light used for aiming or therapy is selectable and can be matched to the size and shape of targeted tissue.

In accordance with one embodiment of the present invention, the PDT apparatus includes an LED array having a plurality of LEDs that are capable of being driven to provide light having a first wavelength for exciting a photosensitizer to provide therapy wherein the LEDs are addressable to light each LED individually or to light a group of LEDs together so that the size and shape of the light emitted by the LED is selectable and can be matched to the size and shape of the targeted tissue. The apparatus also includes one or more optics for receiving light from the LED array and directing the light out of the apparatus.

In accordance with one feature of the present invention, the LED array includes a plurality of microlenses where each microlens is mounted on the LED array to receive light from an individual LED. In one embodiment of the present invention, each microlens is a compound parabolic concentrator.

In accordance with another feature of the present invention, the LED array includes a plurality of rows of LEDs and a plurality of columns of LEDs wherein an LED is individually lit by addressing the row and column of the LED and wherein a group of adjacent LEDs are lit by addressing two or more adjacent rows of the array and one or more columns or by addressing two or more adjacent columns and one or more rows of the array. In one embodiment of the present invention, the anodes of each LED in a row of the array are connected together and the cathodes of each LED in a column are connected together. In this embodiment, a row of the array is addressed by coupling drive current to the row of LEDs and a column of the array is addressed by coupling the column to ground.

In accordance with a further feature of the present invention, the LEDs are positioned in the array so that the distance between a center of a first LED and a center of a second, adjacent LED is equal to the distance between a center of the first LED and a center of a third LED where the third LED is adjacent to both the first and second LEDs and the distance between the center of the first LED and the center of the second LED is equal to the distance between the center of the second LED and the center of the third LED so that the centers of the first, second and third adjacent LEDs form vertices of an equilateral triangle. This embodiment of the LED array allows the array to be tightly packed so as to increase the LED density and pixel density of the array, thereby increasing the irradiance of the array.

In accordance with another feature of the present invention, each LED has a hexagonal shape so as to maximize the packing density of the LEDs and pixels of light generated by the LEDs. The top metallization layer of each of the LEDs is configured to improve current spreading within the active area of the LED while minimizing the amount of light blocked by the metalized lines or traces that provide the current spreading.

In accordance with still another feature of the present invention, the LED array and a filter are controlled to provide an aiming beam having a second wavelength that will not excite the photosensitizer to provide therapy but that will excite the photosensitizer to fluoresce. The LED array and filter, when controlled to provide the aiming beam, allow a physician to select which of the LEDs in the array are to be actuated so as to control the size and shape of the aiming beam and/or therapy light so that when it reaches the targeted tissue the light has substantially the same size and shape as the targeted tissue. The selection of the LEDs may be manual or, alternatively, it may be automatic while allowing the physician to modify the automatic selection.

These and other advantages and novel features of the present invention, as well as details of an illustrated embodiment thereof, will be more fully understood from the following description and drawings.

### BRIEF DESCRIPTION OF THE DRAWINGS

FIG. 1 is a top view of an LED array in accordance with one embodiment of the present invention illustrating the hexagonal active areas of the LEDs of the array;

FIG. 2 is a partial, top view of the LED array illustrating the top metallization layers of the array to provide improved current spreading;

FIG. 3 is an electrical schematic illustrating the connection of the LEDs in one embodiment of the array of the present invention;

FIG. 4 is a partial, side cross-sectional view of the LED array and associated optics;

FIG. 5 is a block diagram of one embodiment of an apparatus of the present invention having an addressable array as depicted in FIG. 1;

FIG. 6 is a flowchart illustrating a method of selecting LEDs of the array for actuation to provide light that substantially matches the size and shape of targeted tissue; and

FIG. 7 is a flowchart illustrating one embodiment of an automatic LED selection process.

### DESCRIPTION OF THE PREFERRED EMBODIMENT

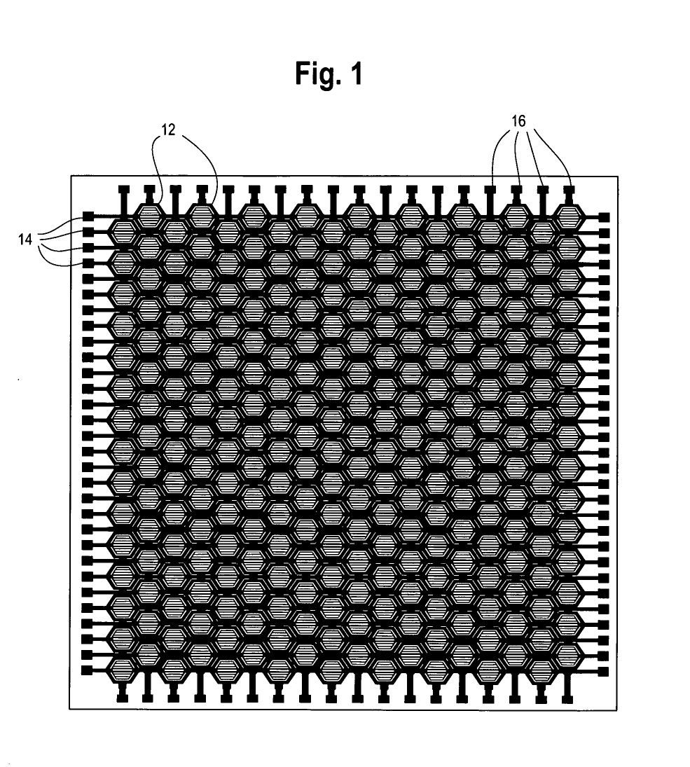

An LED array 10, as shown in FIG. 1, provides light to excite a photosensitizer to diagnose or treat diseased tissue of the eye. The array 10 includes a number of LEDs 12 arranged in rows 14 and columns 16 of the array 10. The LEDs 12 of the array 10 are addressable to light each LED individually or to light a group of LEDs together so that the size and shape of the light emitted by the LED array 10 is selectable and can be matched to the size and shape of diseased eye tissue that is targeted to be treated.

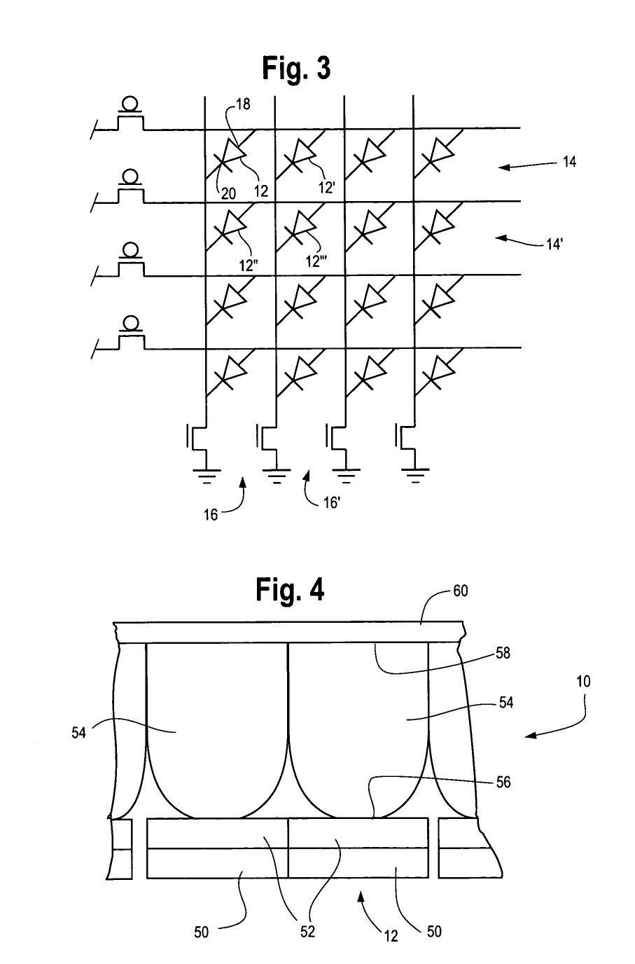

As shown in FIG. 3, the anodes 18 of each LED 12 in a row 14 of the array 10 are connected together. Similarly, the cathodes 20 of each LED 12 in a column 16 of the array 10 are connected together. An individual LED 12 of the array 10 is addressed by coupling drive current to the row of LEDs in which the LED to be lit is located, and by coupling the column, in which the LED to be lit is located, to ground. For example, the LED 12 is addressed so as to light the LED 12 by coupling drive current to the row 14 of LEDs and by connecting the column 16 of LEDs to ground. In order to actuate a group of LEDs, such as the LEDs 12, 12′, 12″, and 12′″, drive current is coupled to the rows 14 and 14′ of the array 10 and the columns 16 and 16′ are connected to ground. The wired configuration of the LEDs in the array 10 as depicted in FIG. 3 maximizes the LED density so that each LED die can be placed as close together as possible while reducing the complexity of addressing as well as reducing the number of wired connections needed to address individual LEDs or groups of LEDs. It is noted, that with this wired configuration of the LED array 10, the LEDs can be addressed to provide light of various solid shapes such as a solid rectangle; but, the LEDs cannot be addressed to light, for example, the outline of a rectangle. However, because diseased tissue of the eye generally has a solid shape, the wired configuration of the array 10 shown in FIG. 3 allows the LEDs of the array 10 to be addressed so as to substantially match the size and shape of almost any diseased or targeted tissue.

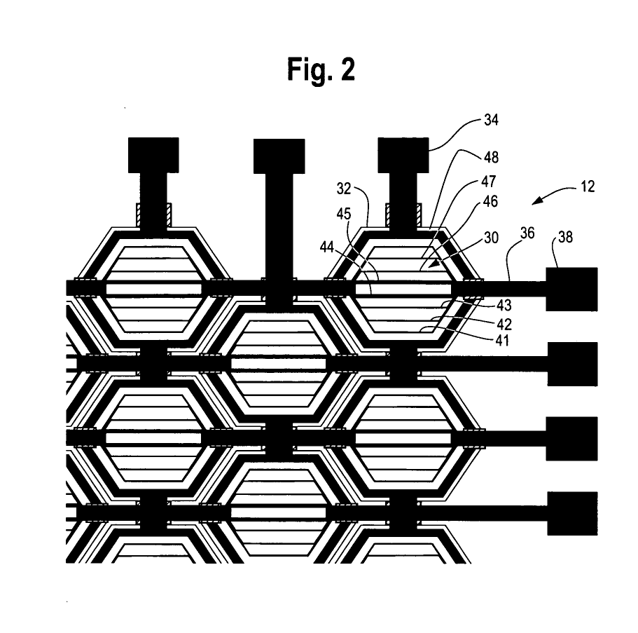

The LEDs in accordance with the present invention are hexagonal in shape so as to maximize the irradiance of each of the individual LEDs of the array and to improve the packing density of the pixels of light generated by the LEDs. Moreover, the hexagonal shape of the LEDs allows a group of adjacent LEDs to be selected to more closely match the shape of targeted tissue than if the LEDs were, for example, square. As shown in detail in FIG. 2, each LED 12 has a hexagonal active area 30 surrounded by a metalized line or trace 32 outlining a hexagon. The metalized trace 32 for the cathode of the LED has a column connection or wire tap 34 for addressing. A horizontal trace or metalized line 36 for the anode of the LED has a row connection or wire tap 38 for addressing. The horizontal trace 36 is split into a number of spaced and parallel sub-traces or metalized lines 41-48 that extend across the active area 30 of the LED 12. The sub-traces 41-48 provide improved current spreading across the active area 30 of the LED 12 while minimizing the amount of light blocked by the metalized lines 41-48. In a preferred embodiment, the thickness of the sub-traces decreases from the middle sub-traces to the sub-traces adjacent the periphery of the active area 30 of the LED 12. As such, the sub-traces, 44 and 45, extending across the middle of the active area 30, are thicker than the sub-traces 41 and 48 that are located near the periphery of the active area. The thicker the trace, the more current is carried across the active area 30 of the LED by the trace. The thicknesses of each of the traces 41-48 are further such that the sum of all of the thicknesses of the sub-traces 41-48 equals the thickness of the horizontal trace 36.

As can be seen from FIGS. 1 and 2, the LED array of the present invention can have twice as many address lines extending across one dimension, for example, the horizontal dimension, as compared to the vertical dimension, without impacting the size of the active area. Further, since the LEDs in a given row of the array are spaced by the LEDs in an adjacent row, the current needed to drive the LEDs in a row is reduced so as to allow the thickness of the horizontal trace for each of the rows to be reduced. Because the thickness of the horizontal trace 36 is reduced, the thicknesses of the sub-traces 41-48 is likewise reduced so as to block less light in the active area 30 of the LED. It is noted that, for macular diseases, the preferred implementation of the LED array uses an 18 column by 30 row array for a total source coverage of approximately 9 mm×9 mm. It is also noted that in a preferred embodiment, drive current is coupled to a row of the array 10 through both a right side connection to the row and a left side connection to the row so that the current load is shared by both sides of the array.

FIG. 4 illustrates a partial cross-sectional view of an LED array 10 and associated array optics. The array optics include one microlens associated with each LED 12 or, alternatively, one microlens associated with multiple adjacent LEDs 12. In a preferred embodiment, each microlens is a compound parabolic concentrator (CPC) 54 that is associated with one LED 12. The CPC 54 is a solid optical element having a first surface or optical aperture 56 for receiving light from the LED 12 wherein the light exits the CPC 54 through a second surface or optical aperture 58 such that the light is nearly collimated. In a preferred embodiment, the emission angle of the CPC 54 is 20° or less. The radius of the first surface 56 of the CPC 54 is smaller than the radius of the second surface 58 of the CPC 18. The first or smaller optical surface 56 of the CPC 54 is mounted on an optimizing refractive index material 52 that overlies the die 50 of the LED 12. In a preferred embodiment, when the CPC 54 is mounted on the LED 12, such that the circular area of the first surface 56 of the CPC 54 forms a circle within the hexagonal active area 30 of the LED die 50 when the LED die 50 is formed as described above with reference to FIG. 2. A holographic diffusing film 60 overlies the CPCs 54 associated with the LED array 10 so that the pixel of light generated by each LED 12 and associated optics appears to be circular rather than hexagonal in shape. Moreover, when adjacent LEDs are lit, the diffusing film 60 eliminates any dark spots between pixels so as to provide a solid block of light.

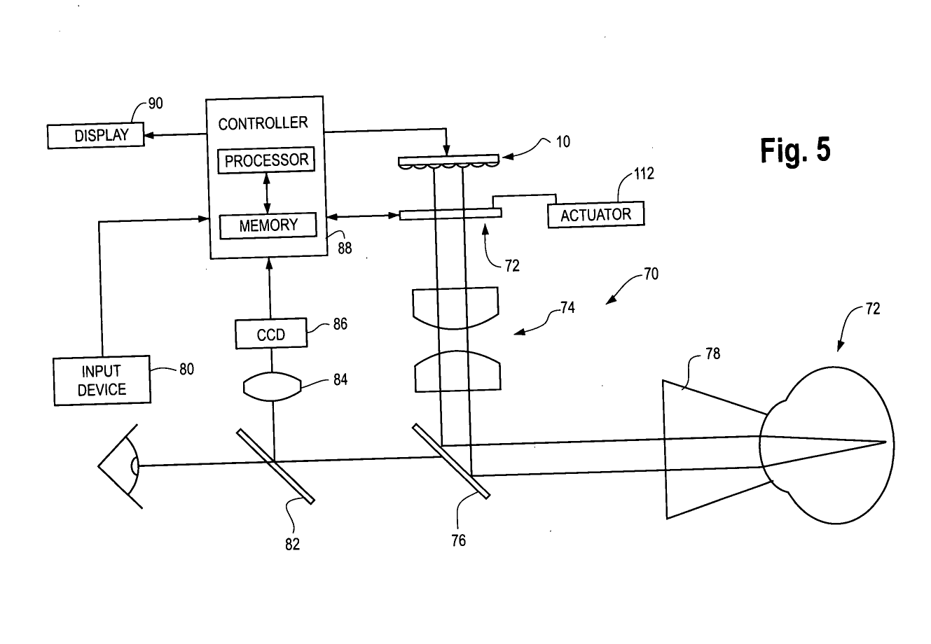

An apparatus 70, as shown in FIG. 5, in accordance with the present invention, directs light from the LED array 10 to a patient's eye 72 for photodynamic therapy. The apparatus 70 preferably includes a movable wavelength selection filter 72 that can be moved into or out of the path of the light from the LED array 10 so as to allow a single light source, i.e. the LED array 10, to be used to provide both therapy light and an aiming beam. For example, when the photosensitizer is talaporphin sodium, the center wavelengths of light for exciting the photosensitizer to provide therapy for diseased tissue is preferably 664 nm. As such, the LEDs 12 of the array 10 are designed to emit light having a constant center wavelength of 664 nm when driven with a high current. In order to produce the aiming beam from the LED array 10, the current driving the LED array 10 is reduced to cause a downward shift in the center wavelength of the light emitted from the array 10 and the filter 72 is moved into the path of the LED array light. The filter 72 is such that it passes a waveband of light that will not excite the photosensitizer for therapy, but the waveband of light will cause the photosensitizer that was preferentially absorbed in the diseased tissue to fluoresce so that the light can be used as an aiming beam. When the photosensitizer is talaporphin sodium, the preferred center wavelength of the aiming beam is 635 nm. After the physician uses the aiming beam to target tissue to be treated, as discussed below, the filter 72 is moved out of the path of the light from the LED array 10 so that light having the therapy wavelength can be directed to the targeted tissue.

The light from the LED array 10 passes through a pair of relay lenses 74 to a safety filter mirror 76. The mirror 76 is a reflector that reflects the light from the LED array 10 out of the apparatus 70 to the patient's 72. In a preferred embodiment, the apparatus 70 is used in conjunction with a contact lens 78 which may be a Meinster high magnification contact lens from Ocular. The safety filter mirror 76 is such that it blocks therapy light reflected from the patient's eye 72 from reaching the physician's eye 80. However, the safety filter mirror 76 allows light that is reflected from the patient's eye 72 and that has the aiming beam wavelength to pass therethrough so as to allow the physician to view tissue that is fluoresced by the aiming beam. The aiming beam can then be used by the physician to select which of the LEDs should remain on for therapy as discussed in detail below. Once the LEDs for therapy are selected, the filter 72 can be moved out of the path of light from the LED array 10 and the current driving the LED array 10 increased to provide the therapy light of a selected size and shape to the targeted eye tissue.

In accordance with one embodiment of the present invention, the apparatus 70 includes an input device 80 that is actuable by the physician so that the physician can select the LEDs of the array 10 to be actuated and lit so that the light that impinges on the targeted tissue of the eye matches the shape and size of the targeted tissue. In one embodiment, the input device 80 may be formed as a keyboard with individual keys mapped to the LEDs of the array 10 such that the physician actuates a key to actuate a corresponding LED of the array 10. In this embodiment, the physician views the interior of the eye through conventional slit lamp viewing optics, not shown, and turns selected LEDs on (or off) using input device 80 so that the LEDs that remain lit generate light matching the shape and size of the targeted tissue. Alternatively, an image of the interior of the eye can be directed, via a semi-reflective mirror 82, through a lens 84 to a CCD camera 86 that captures the image. The captured image can then be input to a controller 88 having a processor and associated memory. The controller 88 controls a display 90 to depict the captured image of the interior of the eye so that the physician can view the image depicting the diseased tissue on the display 90. The processor may also overlay the image of the eye's interior depicted on the display 90 with a grid corresponding to the LED array. The physician can then use an input device 80 in the form of a mouse or joystick that moves a cursor on the display 90 to select various LED represented on the displayed grid. The physician can also move the cursor to outline the targeted tissue to be treated where the outline is mapped to corresponding LEDs of the array 10. In this latter embodiment, the processor actuates not only the LEDs corresponding to the outline, but also the LEDs within the outlined area so as to provide a solid shape of light corresponding to the shape of the targeted tissue.

In accordance with another embodiment of the present invention, the controller 88 can automatically select the LEDs of the array 10 to be actuated and lit to provide therapy and the input device 80 can be used by the physician to modify the automatic selection of the LEDs by the controller 88. In this embodiment, all of the LEDs of the array 10 are initially lit and directed to the eye so that the CCD camera 86 can capture an image of the interior of the eye from the light reflected from the eye to the CCD camera 86. The output of the CCD camera 86 is compared by the controller 88 to a threshold to determine which of the CCD pixels correspond to diseased tissue that is fluorescent due to the interaction of light from the LED array 10 with the photosensitizer that is preferentially absorbed or localized in the diseased tissue. The CCD pixel positions are mapped with the LEDs of the array 10 so that the controller 88 can turn off the LEDs of the array that are associated with the CCD pixels that fall below the threshold, indicating non-diseased tissue. The physician can then view the automatic selection of the LEDs that are actuated by the controller 88 and the physician can modify the automatic selection of the LEDs using the input device 80.

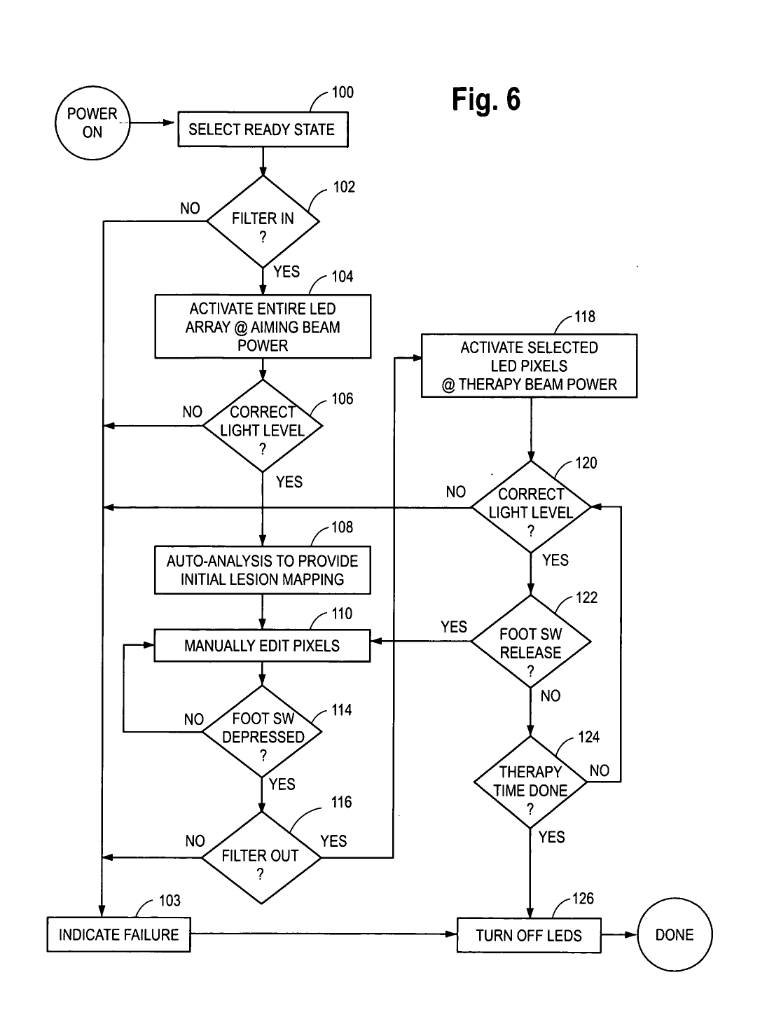

FIG. 6 illustrates a method of operating the device 70 to select the LEDs of the array 10 to be addressed and lit to generate therapy light having a shape and size that matches targeted tissue when the light impinges on the targeted tissue. When power to the apparatus 70 is turned on and a ready state is selected at block 100, the processor of the controller 88 determines at block 102 whether the wavelength selection filter 72 is in the aiming beam position so that when the LED array 10 is turned on, the wavelength selection filter 72 will be in the path of the light from the array 10. The processor can determine the position of the filter 72 by monitoring the position of an actuator 112 that moves the filter 72 into the aiming beam position and out of that position. If the filter is not in the aiming beam position, the processor proceeds from block 102 to block 103 to indicate a failure of the apparatus. If the wavelength selection filter 72 is in the aiming beam position as determined by the processor at block 102, the processor proceeds to block 104 to activate all of the LEDs in the array 10 at the low drive current to provide the aiming beam power and wavelength. At block 108, the processor is responsive to the output of a light sensor, not shown, that detects light levels to determine whether the light level of the array 10 is at the aiming beam level. If the light level of the array 10 is too high, the processor proceeds to block 103 to indicate on the display 90, or the like, a failure and at block 126, the processor turns off the LED array 10. If, however, the processor determines at block 106 that the light level of the array 10 is correct, the processor proceeds to block 108. At block 108, the processor is responsive to the output from the CCD camera 86 for identifying the CCD pixels that are above the threshold indicating that those pixels represent tissue that is fluorescent due to the photosensitizer localized in the diseased tissue. The processor then turns off the LEDs of the array 10 other than those corresponding to the pixel positions representing the diseased tissue to provide an initial mapping of the LEDs that are selected to provide therapy. That is, at block 108, the processor turns off those LEDs that correspond to pixels from the CCD camera 86 that are below the threshold. Thereafter, at block 110, the physician uses the input device 80 to manually edit the selection of the LEDs that were automatically determined by the processor at block 108. When the physician is satisfied that the aiming beam has a shape and size that substantially matches the targeted tissue when the light is impinging on the tissue, the physician actuates a foot switch that controls the actuator 112 to move the filter 72 out of the path of light from the LED array 10. At block 116, the processor determines whether the filter 72 has been moved out of the path of the light from the LED array 10 and, if so, the processor at block 118 increases the drive current to the LED array 10 to activate the selected LED pixels at the therapy beam power and wavelength. Thereafter, at block 120, the processor determines whether the light level of the array 10 is at the correct level to provide therapy light. If the light level is correct, the processor proceeds to block 122 to determine whether the foot switch has been released indicating that the wavelength selection filter 72 is again in the path of the light from the LED array 10. If the foot switch has not been released, the processor proceeds to block 124 to determine whether the therapy time has finished. If not, the processor proceeds back to block 120 to monitor the light level of the LED array 10 to ensure it is correct. When the processor determines at block 124 that the therapy time has finished, the processor proceeds to block 126 to turn off all of the LEDs of the array 10.

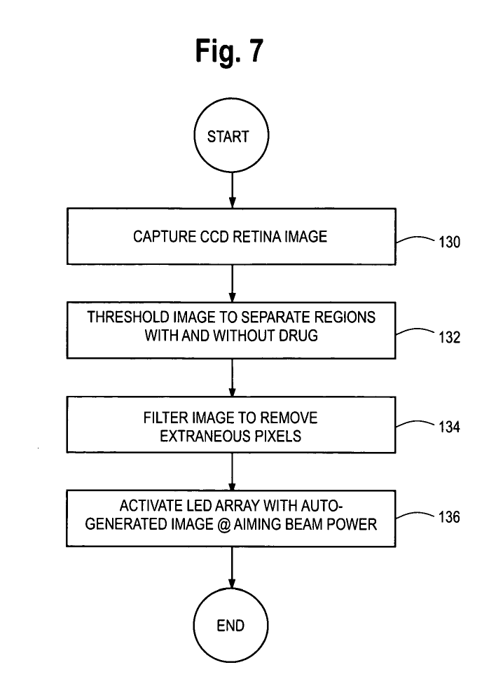

FIG. 7 illustrates the steps of the auto selection process described above. At the start of the process, the CCD camera captures an image of the interior of the eye at a block 130. The processor at block 132 compares the output of the CCD camera to a threshold to separate out the pixels associated with areas of the eye in which the photosensitizer is localized and those areas in which the photosensitizer was not absorbed. At block 134, the processor removes extraneous pixels by filtering the image. Then, at block 136, the processor activates the LEDs of the array 10 that are mapped to the CCD pixel positions indicating the presence of the drug and deactivates the LEDs mapped to the CCD pixel positions indicating an absence of the drug. The LEDs of the array are activated at block 136 at the aiming beam power.

Many modifications and variations of the present invention are possible in light of the above teachings. Thus, it is to be understood that, within the scope of the appended claims, the invention may be practiced otherwise and as described hereinabove. What is claimed and desired to be secured by Letters Patent, is:

1. An apparatus for directing light to an eye to excite a photosensitizer comprising:
an LED array having a plurality of LEDs that are capable of being driven to provide light having a first wavelength for exciting the photosensitizer to provide therapy, the LEDs being addressable to light each LED individually or to light a group of LEDs together so that the size and shape of the light emitted by the LED array is selectable; and
one or more optics for receiving light from the LED array and directing the light out of the apparatus.

2. An apparatus as recited in claim 1 wherein the LED array includes a plurality of microlenses, each microlens being mounted on the array to receive light from an individual LED.

3. An apparatus as recited in claim 2 wherein each microlens is a compound parabolic concentrator.

4. An apparatus as recited in claim 3 wherein the compound parabolic concentrator has an emission angle of 20° or less.

5. An apparatus as recited in claim 3 wherein each compound parabolic concentrator has a first surface that receives light from the LED and a second surface opposite the first surface through which light exits wherein the area of the first surface is smaller than the area of the second surface and the first surface of each compound parabolic concentrator is mounted on an associated LED of the array.

6. An apparatus as recited in claim 5 including a holographic diffusing film of an acrylic material overlying the second surfaces of the compound parabolic concentrators.

7. An apparatus as recited in claim 1 wherein the array includes a plurality of rows of LEDs and a plurality of columns of LEDs wherein an LED is individually lit by addressing the row and column of the LED.

8. An apparatus as recited in claim 7 wherein a group of adjacent LEDs are lit by addressing two or more rows of the array and one or more columns or by addressing two or more columns and one or more rows of the array.

9. An apparatus as recited in claim 7 wherein the anodes of each LED in a row of the array are connected together and the cathodes of each LED in a column of the array are connected together and a row in the array is addressed by coupling drive current to the row of LEDs and a column in the array is addressed by coupling the column to a ground.

10. An apparatus as recited in claim 1 wherein the LEDs are positioned in the array so that the distance between a center of the first LED and a center of the second LED is equal to the distance between the center of the first LED and a center of the third LED that is adjacent to both first and second LEDs and the distance between the center of the first LED and the center of the second LED is equal to the distance between the center of the second LED and the center of the third LED.

11. An apparatus as recited in claim 10 wherein the active area of each LED is hexagonal in shape.

12. An apparatus as recited in claim 10 wherein a metallization layer of an LED in the array includes a metalized line outlining a hexagon.

13. An apparatus as recited in claim 12 wherein a metallization layer of the LED includes a plurality of parallel metalized lines extending across an active area of the LED outlined by the hexagonal metalized line.

14. An apparatus as recited in claim 13 wherein the sum of the thicknesses of the parallel metalized lines extending across the active area of the LED is equal to the thickness of a first metalized line leading into the LED and the parallel metalized lines.

15. An apparatus as recited in claim 14 including a second metalized line connected to the parallel lines and on an opposite side of the LED from the first metalized line, wherein drive current is coupled to the LEDs in a row of the array from a right side connection and from a left side connection.

16. An apparatus as recited in claim 13 wherein the parallel metalized lines extending across a middle of the active area are thicker than the parallel metalized lines extending across the periphery of the active area.

17. An apparatus as recited in claim 1 further comprising a filter movable into and out of the optical path of the light from the LED array, the filter allowing a second wavelength of light to pass that will not excite the photosensitizer to provide therapy, the second wavelength of light providing an aiming beam.

18. An apparatus as recited in claim 17 wherein the therapy light has a center wavelength of approximately 664 nm and the aiming beam has a center wavelength of approximately 635 nm.

19. An apparatus as recited in claim 17 including a current source for driving the LED array with a first current to provide the aiming beam when the filter is in the optical path of the light from the LED array and for driving the LED array with a second current that is greater than the first current to provide the therapy light when the filter is moved out of the optical path of light from the LED array.

20. An apparatus as recited in claim 17 including
optics to allow a physician to view tissue of an eye that has been excited by the aiming beam;
an input device actuable by the physician to provide user inputs for selecting which LEDs of the array are to be used to provide light to tissue targeted by the physician; and
a controller that is responsive to the physician inputs to address one or more LEDs of the array to light or turn off the LEDs in accordance with the user's selection.

21. An apparatus as recited in claim 1 including a system to allow a user to select LEDs to provide therapy light directed to targeted tissue so that the therapy light reaching the targeted tissue has substantially the same size and shape as the targeted tissue.

22. An apparatus as recited in claim 1 including a system to allow a user to select LEDs to provide an aiming beam directed to targeted tissue so that an aiming beam reaching the targeted tissue has substantially the same size and shape as the targeted system.

23. An apparatus as recited in claim 20 including a CCD camera to capture an image of the interior of the patient's eye and wherein the controller is responsive to the output of the CCD camera to automatically provide at least an initial selection of which of the LEDs of the array are to be on or off.

24. An addressable light source for use in an apparatus for directing light to an eye comprising:
an LED array having a plurality of LEDs in a plurality of rows and a plurality of columns of the array, each of the LEDs having an anode and a cathode wherein the anode of the LEDs in a row of the array are connected together, the cathodes of the LEDs in a column are connected together and a row of LEDs is addressed by coupling a drive current to the row and a column of LEDs is addressed by coupling the column to a ground.

25. An apparatus as recited in claim 24 wherein the LEDs are positioned in the array so that the distance between a center of the first LED and a center of the second LED is equal to the distance between the center of the first LED and a center of the third LED that is adjacent to both first and second LEDs and the distance between the center of the first LED and the center of the second LED is equal to the distance between the center of the second LED and the center of the third LED.

26. An apparatus as recited in claim 25 wherein the active area of each LED is hexagonal in shape.

27. An apparatus as recited in claim 24 wherein a metallization layer of an LED in the array includes a metalized line outlining a hexagon.

28. An apparatus as recited in claim 27 wherein a metallization layer of the LED includes a plurality of parallel metalized lines extending across an active area of the LED outlined by the hexagonal metalized line.

29. An apparatus as recited in claim 28 wherein the sum of the thicknesses of the parallel metalized lines extending across the active area of the LED is equal to the thickness of a first metalized line leading into the LED and the parallel metalized lines.

30. An apparatus as recited in claim 29 including a second metalized line connected to the parallel lines and on an opposite side of the LED from the first metalized line, wherein drive current is coupled to the LEDs in a row of the array from a right side connection and from a left side connection.

31. An apparatus as recited in claim 28 wherein the parallel metalized lines extending across a middle of the active area are thicker than the parallel metalized lines extending across the periphery of the active area.

32. An addressable light source for use in an apparatus for directing light to an eye comprising:
an LED array having a plurality of LEDs in a plurality of rows and a plurality of columns of the array, each of the LEDs of the array having a first metalized line outlining a hexagonal active area of the LED and a second metalized line leading to the plurality of sub-lines that are metalized and extend across the active area of the LED, the sum of the thickness of the sub-lines being equal to the thickness of the second metalized line wherein the first metalized lines of the LEDs in a column are connected to allow a column of the array to be addressed via the first metalized line and the second metalized lines of the LEDs in a row are connected to allow a row of the array to be addressed via the second metalized line.

33. An apparatus as recited in claim 32 wherein the sub-lines have spaced parallel sections extending across the active area of an LED.

34. An apparatus as recited in claim 32 wherein the parallel metalized lines extending across a middle of the active area are thicker than the parallel metalized lines extending across the periphery of the active area.

35. An apparatus as recited in claim 32 include a plurality of compound parabolic concentrators, each compound parabolic concentrator being associated with an LED of the array and having a first surface for receiving light from its associated LED and having a second surface through which light exits, the first surface being smaller than the second surface and the first surface being mounted over the active area of the LED.

36. An apparatus as recited in claim 35 wherein the first surface is circular and when overlying the LED forms a circle within the hexagonal active area of the LED.

37. An apparatus as recited in claim 35 wherein each compound parabolic concentrator has an emission angle of 20° or less.

38. An apparatus as recited in claim 35 including a holographic diffusing film of an acrylic material overlying the second surfaces of the compound parabolic concentrators.

39. An apparatus as recited in claim 35 including an optimizing refractive index material disposed between the first surface of each compound parabolic concentrator and the LED.

40. An apparatus for directing light to an eye to excite a photosensitizer comprising:
an LED array having a plurality of LEDs that are capable of being driven to provide light having a first wavelength for exciting the photosensitizer to provide therapy, the LEDs being addressable to light each LED individually or to light a group of LEDs together so that the size and shape of the light emitted by the LED array is selectable;
a filter movable into and out of the optical path of the light from the LED array, the filter when moved into the path of light from the LED array allowing light of a second wavelength to pass, the light of the second wavelength providing an aiming beam;
one or more optics for receiving light from the LED array and directing the light out of the apparatus and for receiving light reflected from a patient's eye, passing light of the second wavelength so that it can be viewed by a physician and for blocking light of the first wavelength; and
an input device operable by a physician to provide inputs to select the LEDs of the array to be turned on or off to provide the aiming beam or therapy.

41. An apparatus as recited in claim 40 including a controller that is responsive to the physician inputs to address one or more of the LEDs of the array to turn the LEDs on or off.

42. An apparatus of claim 41 including a CCD camera to capture an image of the interior of the patient's eye and wherein the controller is responsive to the output of the CCD camera to automatically provide at least an initial selection of which of the LEDs of the array are to be on or off.

43. An apparatus as recited in claim 40 wherein the therapy light has a center wavelength of approximately 664 nm and the aiming beam has a center wavelength of approximately 635 nm.

44. An apparatus as recited in claim 40 including a current source for driving the LED array with a first current to provide the aiming beam when the filter is in the optical path of the light from the LED array and for driving the LED array with a second current that is greater than the first current to provide the therapy light when the filter is moved out of the optical path of light from the LED array.
